(19) United States
(12) Patent Application Publication
(10) Pub. No.: US 2010/0113596 A1
(43) Pub. Date: May 6, 2010

(54) Method for inhibiting liver fibrosis via retinoic acid derivative

(76) Inventor: Kun-Lin Yang, Jiali Town (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/289,811

(22) Filed: Nov. 5, 2008

(51) Int. Cl. A61K 31/203; A61P 1/16

(52) U.S. Cl. 514/559

(57) ABSTRACT

Transforming growth factor-beta1 (TGF-β1) mediates expression of collagen 1A2 (Col 1A2) gene via a synergistic cooperation between Smad2/Smad3 and Sp1, both act on the Col 1A2 gene promoter. The present invention discloses a method for inhibiting liver fibrosis via a retinoic acid derivative primarily extracted from the mycelia of Phlellinus linteus. The retinoic acid derivative can antagonize TGF-β-induced liver fibrosis through regulation of ROS and calcium influx, decreasing the promoter activity of Col 1A2, hindering the translocalization of phosphorylated Smad2/3-Smad4 complex from cytosol into nucleus and inhibiting Sp1 binding activity.

### BACKGROUND OF THE INVENTION

1. Field of the Invention

The present invention relates to a method for inhibiting liver fibrosis via retinoic acid derivative extracted from the mycelia of Phlellinus linteus. In this method, reactive oxygen species (ROS), calcium influx, collagen (Col 1A2) and cycloxygease-2 (Cox-2) induced by transforming growth factor-β (TGF-β) in hepatic stellate cells (HSC-T6), all of these indicators including ROS, Col1A2, and Cox-2 expression are decreased, the hindered translocalization of phosphorylated Smad2/3-Smad4 complex from cytosol into nucleus hindered, and Sp1 binding activity inhibited by treatment with retinoic acid.

2. Brief Description of the Prior Art

Liver fibrosis occurs before liver cirrhosis. When the soft liver is injured in long-period inflammation, the cell fibroblastoma in liver will be stimulated to produce collagen and form fibers accumulating in the liver. The fibers fill empty spaces caused by hepatocellular apoptosis and form fibrosis tissues. Once the liver is subjected to fibrosis and continuously inflames, the fibrosis tissues will accumulate more and more and result in irreversible liver cirrhosis.

Most liver fibrosis is caused by chronic hepatitis, for example, viral hepatitis including hepatitis B and hepatitis C, alcoholic hepatitis, drug-induced hepatitis, autoimmune hepatitis including primary biliary cirohosis or genetic and metabolic deceases including Wilson's disease. In addition, liver fibrosis is possibly sequela though recovered from fulminant hepatitis. After liver fibrosis occurring, structure of the hepatotissue may change to worsen hepatocellular operation and blood flowing. Such problems easily cause high pressure of the liver portal vein and injure the liver more. As the liver has no pain nerve and one needs only 20%˜30% thereof to operate, it's not easy to be conscious of syndromes for the patients. When the fibrosis becomes severe, syndromes such as reddish bumps, palmar erythema and stomach surface varices, moreover, complications such as hematemesis, ascites fluid, hepatic coma and jaundice may appear. In that event, liver function is considerably weak, and liver failure perhaps happens.

According to the statistics of Department of Health, Executive Yuan, R.O.C. (Taiwan), chronic hepatitis and liver cirrhosis were always listed within the first ten reasons of death and resulted in that about four or five thousand people died every year. Most of chronic hepatitis and liver cirrhosis were caused by hepatitis viruses. Furthermore, among people died of cancers, those died of liver cancer were the most, about five or six thousand people in one year, and 90% thereof induced from chronic hepatitis. Therefore, chronic hepatitis perhaps becomes liver cirrhosis after a long time, and 5% of people subjected to liver cirrhosis will be transferred to liver cancer.

So far, liver transplantation is the only solution for treating liver cirrhosis but limited by very few donors. With development of liver fibrosis, particularly during cirrhosis, incidence rate and death rate increase. Therefore, it's an important issue to construct a mode for monitoring fibrosis as early. For example, inhibition, treatment and prevention of liver fibrosis were discussed in J Chin Med 15(4): 257-271, 2004 and U.S. Pat. No. 5,287,835.

The factors causing liver fibrosis include: (1) cells including hepatic cell, sinusoidal endothelial cell, kupffer cell, stellate cell, etc.; (2) cellular factor including transforming growth factor-β (TGF-β), platelet-derived growth factor (PDGF), tumor necrosis factor-α (TNF-α), Interferon-g (IFN-g), interleukin-1a (IL-1a), fibroblast growth factor (FGF), etc.; (3) extracelluar matrix (ECM) including collagen (I, III, IV, V and VI), noncollagens (fibronectin and laminin), proteoglycan (chondroitin sulfate, dermatan sulfate, heparan sulfate, keratan sulfate, heparin and hyaluronic acid).

In the conditions of liver necrosis in inflammation, the above factors promote and constrain each other to develop fibrosis. During this process, activation of hepatic stellate cells (HSC) is a key function. So far, it's considered that activation of HSC include three stages. In the first stage, before inflammation occurs, the liver cells are injured so that extracellular heparan sulfate can not be synthesized and thus contact between the adjacent stellate cells can not be inhibited. The liver cells also secrete some factors to stimulate HSC generation. In the second stage, during inflammation, the activated kupffer cells, sinusoidal endothelial cells, macrophage and decayed blood platelets release TGF-β and PDGF at the apoptosis area to enhance activation of HSC and transferring as fibroblast. In the third stage, after inflammation, fibroblast in the completely transformed form at the static stage will further enhance proliferation and activation of HSC through autocrine and paracrine. In addition, extracelluar matrix, for example, collagen, noncollagen, proteoglycans, are secreted to induce liver fibrosis.

TGF-β can enhance generation of ROS which will change concentration of intracellular calcium ions and thus activation of HSC, expression of α-SMA, accumulation of up-regulated extracelluar matrix are enhanced.

Therefore, if ROS, extracelluar matrix, intracellular calcium ions, translocation of phosphorylated Smad2/3-Smad4 complexes into nucleus and activity of Sp1 binding DNA induced by TGF-β can be inhibited, early liver fibrosis will be inhibited.

### SUMMARY OF THE INVENTION

The object of the present invention is to provide a method for inhibiting liver fibrosis via retinoic acid derivative extracted from the mycelia of Phlellinus linteus.

In this method, reactive oxygen species (ROS), calcium influx, collagen (Col 1A2) and cycloxygease-2 (Cox-2) induced by transforming growth factor-β (TGF-β) in hepatic stellate cells (HSC-T6) are decreased, the translocalization of phosphorylated Smad2/3-Smad4 complex from cytosol into nucleus is hindered, and Sp1 binding activity is inhibited.

The retinoic acid derivative has functions as follows:
1. Protecting the liver cells and slowing down liver fibrosis induced from TGF-β by controlling oxidative stress caused by free radicals and calcium ions entering the cells.
2. Inhibiting transmission of signals which enhance TGF-β-induced fibrosis. The main mechanism of TGF-β-induced fibrosis is the expression of collagen (Col 1A2) which is generated via translocalization of phosphorylated Smad2/3-Smad4 complex into nucleus. The retinoic acid derivative can inhibit the free radical ROS and hinder translocalization of phosphorylated Smad2/3-Smad4 complex into nucleus, and thus inhibit expression of the main collagen of fibrosis.

### BRIEF DESCRIPTION OF THE DRAWINGS

FIG. 1 shows ROS assays on TGF-β1 and PL-treated cells.

FIG. 2 shows [Ca⁺²]ᵢ of HSC-T6 cells pretreated with DMSO or PL after adding TGF-β.

FIG. 3 shows Western blot of α-SMA.

FIG. 4 shows immunocytostaining of HSC-T6 cells treated with (I) TGF-β only, (II) TGF-β+30 nM PL, (III) TGF-β+150 nM PL, and (IV) TGF-β+300 nM PL.

FIG. 5 shows Masson's trichrome staining of ECM and collagen.

FIG. 6 shows effects of PL on Sp1 binding activity.

### DETAILED DESCRIPTION OF THE PREFERRED EMBODIMENTS

In the past, anti-radical was considered an indirect solution for anti-fibrosis, but the tests in vivo were not satisfied. Factually, liver fibrosis is irreversible when able to be clinically diagnosed. In many animal tests for researching liver fibrosis, carbon tetrachloride was used to induce liver fibrosis. However, liver fibrosis could become irreversible before clearly identifying the levels of liver fibrosis. Therefore, the first strategy of the present invention is to construct a mode for detecting early liver fibrosis of mice.

In the present invention, plasmid (pPK9a) was injected into the mouse tail vein in a short duration of 5˜7 seconds according to the hydrodynamics-based transfection protocol. The results indicate that the mice fed with ZnSO₄ had the most obvious performance in TGF-β. Content of TGF-β in serum reached 900˜600 pg/ml after 48 hours. The downstream protein (p-Smad2/3-Smad4 and Sp1) also increased, and TGF-β and Sp1 recovered to the normal values. TGF-β is the most obvious cytokines in early fibrosis and reversible in this mode, so that the early fibrosis mode of mice is constructed.

Additionally, many researches indicated that transformation growth factor-β (TGF-β) can up-regulate expression of collagen 1A2 (Col 1A2) via reactive oxygen species (ROS) related to inflammation of cycloxygease-2 (Cox-2) which results in activation of HSC and thus enhances fibrosis. Therefore, the second strategy of the present invention is to construct a screening platform in which the promoters including ROS, Cox-2 and Col 1A2 are used to evaluate effects of anti-liver fibrosis.

In the present invention, the retinoic acid derivative has the following structural formula

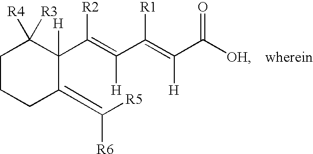

R₁, R₃ and R₄ are independently one of OH, SH₂ and NH₂; and
R₂, R₅ and R₆ are independently one of OH, SH₂, NH₂ and CH₃.

In the present invention, TGF-β-induced ROS, concentration of intracellular calcium ions, [Ca²⁺]ᵢ, are used to evaluate expression of the downstream proteins of TGF-β, i.e., influences of retinoic acid derivative on p-Smad2/3, Sp1 and nonproteins such as ROS and [Ca²⁺]ᵢ. The influence of retinoic acid derivative on ending collagen is measured, too. As the extract of Phlellinus linteus with ethanol performs strong anti-oxidation, the extract is further purified to find retinoic acid derivative thereof. The retinoic acid derivative is used in the preferred embodiments for decreasing expression of TGF-β-induced proteins such as p-Smad2/3, collagen, and nonproteins such as ROS and [Ca²+]ᵢ so as to verify effects of the retinoic acid derivative in inhibition of early liver fibrosis.

### [Materials]

### 1. HSC-T6 Cell Line

In vitro studies were performed in HSC-T6 cells, a generous gift of Prof. S.L. Friedman of the Mount Sinai School of Medicine (NY, USA).

### 2. Chemical

A retinoic acid derivative extracted from the mycelia of Phlellinus linteus was isolated and purified by the Genefarm Biotech Co., Taiwan. The retinoic acid derivative (hereinafter denoted as PL) was dissolved in dimethyl sulfoxide (DMSO). Its formula is shown as follows

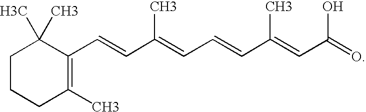

### 3. Mice

All procedures of animal handling were approved by the Institutional Animal Care and Use committee of National Cheng Kung University.

4. cDNA Construct

TGF-β1 cDNA was constructed in pPK9a vector and a gift from Professor Kondaiah.

### [Methods]

The eight-week-old BALB/c mice were fed ad libitum standard laboratory chow and water with or without 25 mM ZnSO₄ plus 5% sucrose. PL was given intraperitoneally (i.p.) at 30, 150 or 300 μg/kg. The animals were hydrodynamically injected with pPK9a plasmid containing TGF-β1 gene through tail vein and simultaneously fed with ZnSO₄(25 mM) and PL as reported. After 48 h, the mice were sacrificed for liver sections.

### [Measurements]

### 1. Measurement of ROS and α-SMA

Cells were incubated with 2 ng/ml of TGF-β and PL for 5 hours and analyzed for the oxidation state using 7-dichlorodihydrofluorescein diacetate (DCFH-DA) as a fluorogenic probe and flow cytometer (FACSCalibur). The cells were recovered with 0.01% trypsin and the cellular fluorescence intensity was measured after 30 min incubation of 5 μM DCFHDA. For each analysis, 10,000 events were recorded. Cells were collected in 0.01% trypsin, fixed with 3.7% formaldehyde, permeabilized with 0.02% Triton X-100 in PBS, and stained for a-SMA as described previously. For detecting a-SMA using flow cytometry, cells were treated with 2 ng/ml of TGF-β and PL for 12 hours and immunostained with α-SMA-specific antibody (Santa Cruz) and FITC-conjugated secondary antibody (Molecular Probe). Then the cells were recovered and subjected to flow cytometer, and PL can effectively eliminate radicals as ROS decreased with increasing dosages of PL, as referred to FIG. 1.

2. Measurement of Cytosolic Ca²⁺ ([Ca²⁺]ᵢ)

Trypsinized cells (10⁶/ml) were recovered 4 h after culture with PL and loaded with 2 μM fura-2/acetoxy methyl (fura-2/AM) for 30 min at 25° C. before stimulating with TGF-β (2ng/ml). Cells were gently shaken for several seconds every 10 min to prevent re-attachment. Cells were washed and resuspended in Ca²⁺-containing medium. Fura-2 fluorescence measurements were performed in a water-jacketed cuvette with continuous stirring at 25° C. Fluorescence was monitored with a Hitachi F-2500 fluorescence spectrophotometer by recording excitation signals at), and 380 nm and emission signals at 510 nm at 1-s intervals. It is found that a trace amount of PL is sufficient enough to block-[Ca²⁺]_{I} as referred to FIG. 2.

### 3. Hydrodynamics-Based Transfection of TGF-β1 Gene

Ten milligrams of plasmid (pPK9a) was dissolved in 3.0 ml Ringer's solution and injected in bolus into the tail vein in a short duration of 5˜7 seconds according to the hydrodynamics-based transfection protocol as described. ZnSO₄(25 mM) was dissolved in the drinking water to activate the metallothionein promoter and stimulate TGF-β1 expression.

### 4. Histological and Immunohistochemical Analysis

Mouse liver tissues were embedded in an optimal cutting temperature compound (Miles Inc.) and frozen in liquid nitrogen. Five millimeter cryosections were made by using cryostats (Leica Ch4 1800]. The sections were fixed with cold acetone and endogenous peroxidase was inhibited by 3% H₂O₂ in PBS. The sections were then incubated with 5% blocking serum (normal serum of the species of the secondary antibody). For modeling the negative control sections, the primary antibodies were substituted for the appropriate classes and isotypes of normal immunoglobulins (Igs). Controls for nonspecific binding of the secondary antibody were performed by replacing the solutions of the first step with PBS. For detecting α-SMA, a mouse monoclonal antibody (Santa Cruz) was used. Signals were visualized by anti-mouse HRP-conjugated secondary antibody, and 3,3′-diaminobenzidine substrate (Vector Laboratories). All sections were viewed under a microscope (Leica Mikrosysteme Vertrieb GmbH), as referred to FIG. 4.

### 5. Masson's Trichrome Staining

Liver specimens were preserved in 4% paraformaldehyde in PBS and dehydrated in a graded alcohol series. Following xylene treatment, the specimens were embedded in paraffin blocks and cut into 5 μm-thick sections that were stained with Masson's trichrome as shown in FIG. 5.

### 6. Western Blot

Liver tissues were homogenized in RIPA buffer and centrifuged as described; supernatant was taken as a whole-cell lysate. These proteins were electrophoresed on a 12% SDS-polyacrylamide gel, transferred by electroblotting to a PVDF membrane, and visualized by immunostaining. Anti-GAPDH (Santa Cruz) and anti-α-SMA antibody (Lab Vision) were used as the primary antibodies. Secondary antibodies were conjugated with horseradish peroxidase (Bio-Rad). The signals were visualized by an enhanced chemiluminescence system (Amersham).

### [Results]

### 1. Effect of PL on Intracellular ROS Production

ROS intermediates act as signaling molecules of TGF-β in regulation of inflammatory and fibrotic responses. Therefore, first select marker was to determine whether PL could be an antioxidant to block TGF-β-induced ROS in HSC-T6 cells. The intracellular oxidation state of the cell was analyzed by flow cytometry, when ROS were induced by TGF-β1 (2 ng/ml). Pretreatment of the cells with 30˜300 nM PL could sufficiently eliminate the ROS generated by TGF-β1 in a dose-dependent manner.

As shown in FIG. 1, the solid lines of (I), (II), (III) and (IV) represent contents of ROS in cells during liver fibrosis. The dotted lines of (I), (II), (III) and (IV) respectively represent contents of ROS in normal liver cells, treated with 30 nm PL, treated with 150 nm PL and treated with 300 nm PL. These results indicate that PL can effectively eliminate radicals as ROS decreased with increasing dosages of PL.

2. PL Directly Decreased the Level of Intracellular Calcium ([Ca²⁺]ᵢ) and α-SMA Expression in HSC-T6 Cells

Generation of ROS has been reported to be a potential mechanism for alteration of intracellular calcium and upregulated ECM protein accumulation. Therefore, we examined whether PL could change the concentration of [Ca²⁺]ᵢ in TGF-β treated cells. Activation of HSC, the main source of liver collagen, involves the induction of α-SMA, conversing from quiescent to proliferation, and secretion of collagen. After treatment with PL, TGF-β1 was added to HSC-T6 cells and the [Ca²⁺]ᵢ was measured immediately (FIG. 2). PL decreased the protein levels of α-SMA, which was detected by both flow cytometry and Western blot (FIG. 3). [Ca²⁺]ᵢ and α-SMA expression were significantly reduced in HSC-T6 as compared with positive control as shown in FIG. 3. The results imply that a trace amount of PL is sufficient enough to block-[Ca²⁺]ᵢ and down-regulate α-SMA expression.

### 3. Effects of PL on the Collagen (Col 1A2) and α-SMA Expression in TGF-β1 Transferred Fibrotic Mice

It has been shown that TGF-β plays a major role in fibrogenesis via the activation of HSC. After the HSC-T6 cells were treated with TGF-β and PL for 4 hours, the cells were subjected to immunostaining and observed under a fluorescence microscopy, as shown in FIG. 4.

FIG. 4 shows HSC-T6 cells treated with (I) TGF-β only, (II) TGF-β+30 nM PL, (III) TGF-β+150 nM PL, and (IV) TGF-β+300 nM PL for 4 hours and cells were subjected to immunostaining and observed under a fluorescence microscopy.

These results indicated that the PL can effectively decrease expression of Col 1A2 to 0.001%. PL also markedly inhibited α-SMA expression in liver tissue as compared with the positive controls, as shown in FIG. 3. Activation of HSC-T6 cells can be evaluated according to expression of α-SMA, and the results indicated that expression of A-SMA decreased with PL increasing. That is, PL inhibited activation of HSC-T6 and TGF-β1-induced collagens.

4. Effects of PL on TGF-β-Induced p-Smad2/3 and Smad4 Translocation Into Nucleus

Translocation of phosphorylated Smad complexes into nucleus is one of the key steps in signal transduction of the liver fibrosis. In activated HSC there is an elevated accumulation of p-Smad2/3 and Smad4 in nucleus. To understand how PL regulates Smad signaling pathway, we first determined the amount of Smad complexes using Western blot. We found that PL decreased the amount of p-Smad2/3 in the cell nucleus in a dose-dependent manner. A further study using immunocytostaining to detect the distribution of Smads, we found that PL decreased the transport of p-Smad2/3 into cell nucleus also in a dose-dependent manner.

### 5. PL Decreased Sp1 Binding Activity

It has been demonstrated that TGF-β1 acts as a strong activator of ECM accumulation to stimulate the Col 1A2 gene expression by inducing the binding of a Sp1- and p-Smad2/3-Smad4-containing complex to Col 1A2 upstream promoter region. Since Sp1 is a critical mediator of Col 1A2 expression, we examined the effects of PL on Sp1 binding activity in pPK9a-transferred mice fed with ZnSO₄ water. As shown in FIG. 6, the EMSA study indicated that TGF-β1 increased Sp1 binding activity in the liver of transgenic mouse (lane 1 vs. 6) but the increase was reversed by PL treatment (lane 2-4). These results suggest that PL inhibits TGF-β-induced Col 1A2 promoter activity through blocking ROS and calcium influx as well as impeding Sp1 binding and translocalization of pSmad 2/3-Smad4 complex into nucleus.

What is claimed is:

1. A method for inhibiting liver fibrosis via a retinoic acid derivative, in which the retinoic acid derivative is applied to inhibition of early liver fibrosis.

2. The method of claim 1, wherein the retinoic acid derivative is extracted from the mycelia of Phlellinus linteus.

3. The method of claim 1, wherein the retinoic acid derivative has the following structural formula: 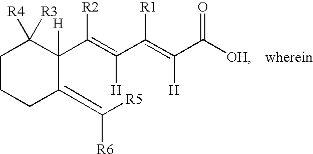
R₁, R₃ and R₄ are independently one of OH, SH₂ and NH₂; and
R₂, R₅ and R₆ are independently one of OH, SH₂, NH₂ and CH₃.
